(19) United States
(12) Patent Application Publication
(10) Pub. No.: US 2022/0127262 A1
(43) Pub. Date: Apr. 28, 2022

(54) CRYSTALLINE FORM OF A CDK INHIBITOR

(71) Applicant: Pfizer Inc., New York, NY (US)

(72) Inventors: Douglas Carl Behenna, San Juan Capistrano, CA (US); Martha Alicia Ornelas, San Diego, CA (US)

(73) Assignee: Pfizer Inc., New York, NY (US)

(21) Appl. No.: 17/423,143

(22) PCT Filed: Jan. 13, 2020
(86) PCT No.: PCT/IB2020/050240

§ 371 Date: Jul. 15, 2021

Related U.S. Application Data
(60) Provisional application No. 62/793,516, filed on Jan. 17, 2019.
(60) Provisional application No. 62/949,990, filed on Dec. 18, 2019.

(51) Int. Cl. C07D 471/04; A61P 35/00

(52) U.S. Cl. CPC C07D 471/04; C07B 2200/13; A61P 35/00

(57) ABSTRACT

The invention relates to a crystalline form of 6-(difluoromethyl)-8-[(1R,2R)-2-hydroxy-2-methylcyclopentyl]-2-{[1-(methylsulfonyl) piperidin-4-yl]amino}pyrido[2,3-d]pyrimidin-7(8H)-one (PF-06873600) free base (Form 1). The invention also relates to pharmaceutical compositions comprising this crystalline form and to methods of using the crystalline form and such compositions for the treatment of abnormal cell growth, such as cancer, in a mammal Formula (I):

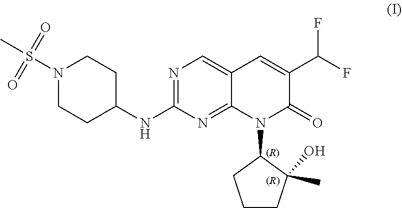

### CROSS-REFERENCE TO RELATED APPLICATIONS

Not applicable.

### BACKGROUND OF THE INVENTION

### Field of the Invention

This invention relates to an anhydrous crystalline form of 6-(difluoromethyl)-8-[(1R,2R)-2-hydroxy-2-methylcyclopentyl]-2-{[1-(methylsulfonyl)piperidin-4-yl]amino}-pyrido[2,3-d]pyrimidin-7(8H)-one (PF-06873600) free base (Form 1), to pharmaceutical compositions comprising Form 1, and to methods of using Form 1 and such compositions in the treatment of abnormal cell growth, such as cancer, in mammals.

### Description of Related Art

The compound PF-06873600 is a potent inhibitor of CDK2, CDK4 and CDK6 having the formula (I):

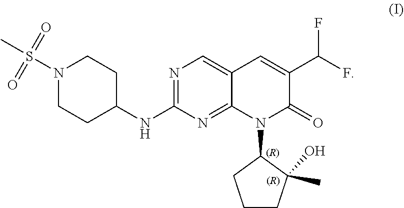

Preparation of PF-06873600 free base is disclosed in International Patent Publication No. WO 2018/033815 and in U.S. Pat. No. 10,233,188. A process for preparing PF-06873600 is described in International Patent Application No. PCT/IB2019/058042. The contents of each of the foregoing documents are incorporated herein by reference in their entirety.

The present invention provides an anhydrous crystalline form of PF-06873600 free base (Form 1) having desirable properties, such as high crystallinity, high purity, low hygroscopicity, favorable dissolution and mechanical properties, and/or favorable stability.

### BRIEF SUMMARY OF THE INVENTION

In one aspect, the invention provides a novel crystalline form of PF-06873600 free base (Form 1). Form 1 of PF-06873600 free base is characterized by one or more of the following methods: (1) powder X-ray diffraction (PXRD) (2θ); (2) Raman spectroscopy (cm⁻¹); (3) ¹³C solid state NMR spectroscopy (ppm); or (4) ¹⁹F solid state NMR spectroscopy (ppm).

In a first aspect, the invention provides PF-06873600 free base (Form 1), which is characterized by having:

(1) a powder X-ray diffraction (PXRD) pattern (2θ) comprising: (a) one, two, three, four, five or more than five peaks selected from the group consisting of the peaks in Table 1 in °2θ±0.2 °2θ; (b) one, two, three or four peaks selected from the group consisting of the characteristic peaks in Table 1 in °2θ±0.2 °2θ; or (c) peaks at 2θ values essentially the same as shown in FIG. 1; or

(2) a Raman spectrum comprising: (a) one, two, three, four, five, or more than five wavenumber (cm⁻¹) values selected from the group consisting of the values in Table 2 in cm⁻¹±2 cm⁻¹; (b) one, two, three, four or five wavenumber (cm⁻¹) values selected from the group consisting of the characteristic values in Table 2 in cm⁻¹±2 cm⁻¹; or (c) wavenumber (cm⁻¹) values essentially the same as shown in FIG. 2; or

(3) a ¹³C solid state NMR spectrum (ppm) comprising: (a) one, two, three, four, five, or more than five resonance (ppm) values selected from the group consisting of the values in Table 3 in ppm±0.2 ppm; (b) one, two, three, four or five resonance (ppm) values selected from the group consisting of the characteristic values in Table 3 in ppm±0.2 ppm; or (c) resonance (ppm) values essentially the same as shown in FIG. 3; or

(4) a ¹⁹F solid state NMR spectrum (ppm) comprising: (a) one or two resonance (ppm) values selected from the group consisting of the values in Table 4 in ppm±0.2 ppm; or (b) resonance (ppm) values essentially the same as shown in FIG. 4;

or a combination of any two, three or four of the foregoing embodiments (1)(a)-(c), (2)(a)-(c), (3)(a)-(c), or (4)(a)-(b), provided they are not inconsistent with each other.

In another aspect, the invention further provides a pharmaceutical composition comprising PF-06873600 free base (Form 1), according to any of the embodiments described herein, and a pharmaceutically acceptable carrier or excipient.

In another aspect, the invention provides a method of treating abnormal cell growth in a mammal, including a human, comprising administering to the mammal a therapeutically effective amount of PF-06873600 free base (Form 1).

In another aspect, the invention provides a method of treating abnormal cell growth in a mammal, comprising administering to the mammal a therapeutically effective amount of a pharmaceutical composition comprising PF-06873600 free base (Form 1), according to any of the aspects or embodiments described herein.

In another aspect, the invention provides use of PF-06873600 free base (Form 1), or a pharmaceutical composition comprising the PF-06873600 free base (Form 1), according to any of the aspects or embodiments described herein, in a method of treating abnormal cell growth in a mammal.

In yet another aspect, the invention provides use of PF-06873600 free base (Form 1), according to any of the aspects or embodiments described herein, in the manufacture of a medicament for the treatment of abnormal cell growth in a mammal.

In frequent embodiments, the abnormal cell growth is cancer. In one embodiment, the abnormal cell growth is cancer mediated by CDK2, CDK4 and/or CDK6. In some such embodiments, the abnormal cell growth is cancer mediated by CDK2. In other such embodiments, the abnormal cell growth is cancer mediated by CDK4 and/or CDK6 In other embodiments, the abnormal cell growth is cancer mediated by CDK2 and CDK4/6. In some embodiments, the cancer is characterized by amplification or overexpression of CCNE1 and/or CCNE2.

In some embodiments, the abnormal cell growth is cancer, wherein the cancer is selected from the group consisting of breast cancer, ovarian cancer, bladder cancer, uterine cancer, prostate cancer, lung cancer (including NSCLC, SCLC, squamous cell carcinoma or adenocarcinoma), esophageal cancer, head and neck cancer, colorectal cancer, kidney cancer (including RCC), liver cancer (including HCC), pancreatic cancer, stomach (i.e., gastric) cancer and thyroid cancer. In further embodiments of the methods provided herein, the cancer is selected from the group consisting of breast cancer, ovarian cancer, bladder cancer, uterine cancer, prostate cancer, lung cancer, esophageal cancer, liver cancer, pancreatic cancer and stomach cancer. In some such embodiments, the cancer is characterized by amplification or overexpression of CCNE1 and/or CCNE2.

In other embodiments, the cancer is breast cancer, including, e.g., ER-positive/HR-positive breast cancer, HER2-negative breast cancer; ER-positive/HR-positive breast cancer, HER2-positive breast cancer; triple negative breast cancer (TNBC); or inflammatory breast cancer. In some embodiments, the breast cancer is endocrine resistant breast cancer, trastuzumab resistant breast cancer, or breast cancer demonstrating primary or acquired resistance to CDK4/CDK6 inhibition. In some embodiments, the breast cancer is advanced or metastatic breast cancer. In some embodiments of each of the foregoing, the breast cancer is characterized by amplification or overexpression of CCNE1 and/or CCNE2.

### BRIEF DESCRIPTION OF THE SEVERAL VIEWS OF THE DRAWINGS

FIG. 1. PXRD pattern of PF-06873600 free base (Form 1).

FIG. 2. FT-Raman spectrum of PF-06873600 free base (Form 1).

FIG. 3. Carbon CPMAS spectrum of PF-06873600 free base (Form 1) (# indicates spinning sidebands).

FIG. 4. Fluorine MAS spectrum of PF-06873600 free base (Form 1) (# indicates spinning sidebands).

FIG. 5. Configuration of flow reactor in Example 1.

### DETAILED DESCRIPTION OF THE INVENTION

The present invention may be understood more readily by reference to the following detailed description of the embodiments of the invention and the Examples included herein. It is to be understood that the terminology used herein is for the purpose of describing specific embodiments only and is not intended to be limiting. It is further to be understood that unless specifically defined herein, the terminology used herein is to be given its traditional meaning as known in the relevant art.

As used herein, the singular form “a”, “an”, and “the” include plural references unless indicated otherwise. For example, “a” substituent includes one or more substituents.

The term “about” means having a value falling within an accepted standard of error of the mean, when considered by one of ordinary skill in the art.

As used herein, the term “essentially the same” means that variability typical for a particular method is taken into account. For example, with reference to X-ray diffraction peak positions, the term “essentially the same” means that typical variability in peak position and intensity are taken into account. One skilled in the art will appreciate that the peak positions (2θ) will show some variability, typically as much as ±0.2°. Further, one skilled in the art will appreciate that relative peak intensities will show inter-apparatus variability, as well as variability due to the degree of crystallinity, preferred orientation, prepared sample surface, and other factors known to those skilled in the art and should be taken as qualitative measures only. Similarly, Raman spectrum wavenumber (cm⁻¹) values show variability, typically as much as ±2 cm⁻¹, while ¹³C and ¹⁹F solid state NMR spectrum (ppm) show variability, typically as much as ±0.2 ppm.

The term “crystalline” as used herein, means having a regularly repeating arrangement of molecules or external face planes. Crystalline forms may differ with respect to thermodynamic stability, physical parameters, x-ray structure and preparation processes.

The term “anhydrous” as used herein, refers to a crystalline form that only contains the active pharmaceutical ingredient (API) as part of its crystalline lattice.

The invention described herein may be suitably practiced in the absence of any element(s) not specifically disclosed herein. Thus, for example, in each instance herein any of the terms “comprising”, “consisting essentially of”, and “consisting of” may be replaced with either of the other two terms.

In one aspect, the invention provides PF-06873600 free base (Form 1). The methods described herein provide PF-06873600 free base (Form 1) which is substantially pure and free of alternative forms.

As described herein, Form 1 was characterized by PXRD, Raman spectroscopy, and ¹³C and ¹⁹F solid state NMR spectroscopy. Such crystalline forms may be further characterized by additional techniques, such as Fourier-Transform InfraRed Spectroscopy (FTIR), Differential Scanning Calorimetry (DSC), Thermogravimetric Analysis (TGA) or Differential Thermal Analysis (DTA).

In some embodiments of each of the aspects of the invention, PF-06873600 free base (Form 1) is characterized by its powder X-ray diffraction (PXRD) pattern. In other embodiments of each of the aspects of the invention, PF-06873600 free base (Form 1) is characterized by its Raman spectrum. In other embodiments of each of the aspects of the invention, PF-06873600 free base (Form 1) is characterized by its ¹³C solid state NMR spectrum. In still other embodiments of each of the aspects of the invention, PF-06873600 free base (Form 1) is characterized by its ¹⁹F solid state NMR spectrum.

In further embodiments, PF-06873600 free base (Form 1) is characterized by a combination of two, three or four of these methods. Exemplary combinations including two or more of the following are provided herein: powder X-ray diffraction (PXRD) pattern (2θ); Raman spectrum wavenumber values (cm⁻¹); ¹³C solid state NMR spectrum (ppm); or ¹⁹F solid state NMR spectrum (ppm).

It will be understood that various combinations of two, three or four techniques may be used to uniquely characterize PF-06873600 free base (Form 1) disclosed herein. In some embodiments PF-06873600 free base (Form 1) is characterized by PXRD and Raman. In other embodiments PF-06873600 free base (Form 1) is characterized by PXRD and ¹³C solid state NMR. In other embodiments PF-06873600 free base (Form 1) is characterized by PXRD and ¹⁹F solid state NMR. In other embodiments PF-06873600 free base (Form 1) is characterized by ¹⁹F solid state NMR and Raman. In other embodiments PF-06873600 free base (Form 1) is characterized by ¹⁹F solid state NMR and ¹³C solid state NMR. In other embodiments PF-06873600 free base (Form 1) is characterized by PXRD, Raman and ¹³C solid state NMR. In other embodiments PF-06873600 free base (Form 1) is characterized by PXRD, Raman and ¹⁹F solid state NMR.

In one embodiment, PF-06873600 free base (Form 1) has a PXRD pattern comprising one or more peaks at 2θ values selected from the group consisting of: 6.9, 9.6, 18.3 and 22.1 °2θ±0.2 °2θ. In another embodiment, PF-06873600 free base (Form 1) has a PXRD pattern comprising two or more peaks at 2θ values selected from the group consisting of: 6.9, 9.6, 18.3 and 22.1 °2θ±0.2 °2θ. In another embodiment, PF-06873600 free base (Form 1) has a PXRD pattern comprising three or more peaks at 2θ values selected from the group consisting of: 6.9, 9.6, 18.3 and 22.1 °2θ±0.2 °2θ.

In one embodiment, PF-06873600 free base (Form 1) has a PXRD pattern comprising peaks at 2θ values of: 9.6, 18.3 and 22.1 °2θ±0.2 °2θ. In some such embodiments, Form 1 has a PXRD pattern further comprising a peak at the 2θ value of: 6.9 °2θ±0.2 °2θ.

In another embodiment, PF-06873600 free base (Form 1) has a PXRD pattern comprising a peak at a 2θ value of: 9.6 °2θ±0.2 °2θ. In another embodiment, Form 1 has a PXRD pattern comprising a peak at a 2θ value of: 18.3 °2θ±0.2 °2θ. In another embodiment, Form 1 has a PXRD pattern comprising a peak at a 2θ value of: 22.1 °2θ±0.2 °2θ. In another embodiment, Form 1 has a PXRD pattern comprising a peak at a 2θ values of: 6.9 °2θ±0.2 °2θ. In some such embodiments, the PXRD pattern further comprises one or more additional peaks at 2θ values selected from the group consisting of the peaks in Table 1.

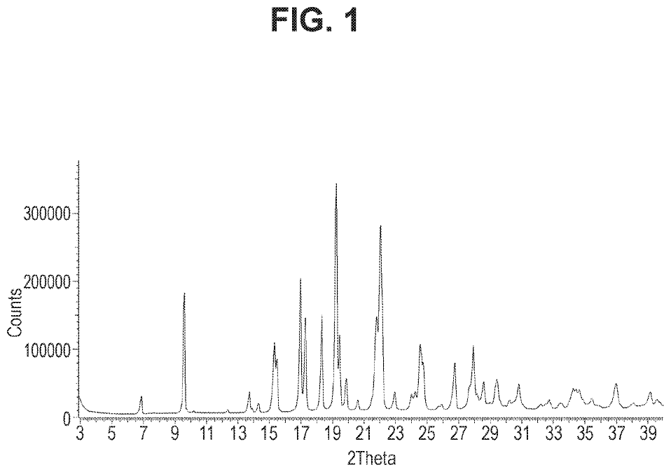

In specific embodiments, PF-06873600 free base (Form 1) has a PXRD pattern comprising: (a) one, two, three, four, five, or more than five peaks selected from the group consisting of the peaks in Table 1 in °2θ±0.2 °2θ; (b) one, two, three or four peaks selected from the group consisting of the characteristic peaks in Table 1 in °2θ±0.2 °2θ; or (c) peaks at 2θ values essentially the same as shown in FIG. 1.

In one embodiment, PF-06873600 free base (Form 1) has a Raman spectrum comprising one or more wavenumber (cm⁻¹) values selected from the group consisting of: 1254, 1528, 1589, 1626 and 1673 cm⁻¹±2 cm⁻¹. In another embodiment, PF-06873600 free base (Form 1) has a Raman spectrum comprising two or more wavenumber (cm⁻¹) values selected from the group consisting of: 1254, 1528, 1589, 1626 and 1673 cm⁻¹±2 cm⁻¹. In another embodiment, PF-06873600 free base (Form 1) has a Raman spectrum comprising three or more wavenumber (cm⁻¹) values selected from the group consisting of: 1254, 1528, 1589, 1626 and 1673 cm⁻¹±2 cm⁻¹.

In another embodiment, PF-06873600 free base (Form 1) has a Raman spectrum comprising wavenumber (cm⁻¹) values of: 1589, 1626 and 1673 cm⁻¹±2 cm⁻¹. In some such embodiments, Form 1 has a Raman spectrum further comprising a wavenumber (cm⁻¹) value of: 1254 cm⁻¹±2 cm⁻¹. In some such embodiments, Form 1 has a Raman spectrum further comprising a wavenumber (cm⁻¹) value of: 1528 cm⁻¹±2 cm⁻¹. In another embodiment, PF-06873600 free base (Form 1) has a Raman spectrum comprising wavenumber (cm⁻¹) values of: 1254, 1528, 1589, 1626 and 1673 cm⁻¹±2 cm⁻¹. In another embodiment, PF-06873600 free base (Form 1) has a Raman spectrum comprising a wavenumber (cm⁻¹) value of: 1589 cm⁻¹±2 cm⁻¹. In another embodiment, Form 1 has a Raman spectrum comprising a wavenumber (cm⁻¹) value of: 1626 cm⁻¹±2 cm⁻¹. In another embodiment, Form 1 has a Raman spectrum comprising a wavenumber (cm⁻¹) value of: 1673 cm⁻¹±2 cm⁻¹. In some such embodiments, Form 1 has a Raman spectrum further comprising the wavenumber (cm⁻¹) value of: 1254 cm⁻¹±2 cm⁻¹. In some such embodiments, Form 1 has a Raman spectrum further comprising the wavenumber (cm⁻¹) value of: 1528 cm⁻¹±2 cm⁻¹.

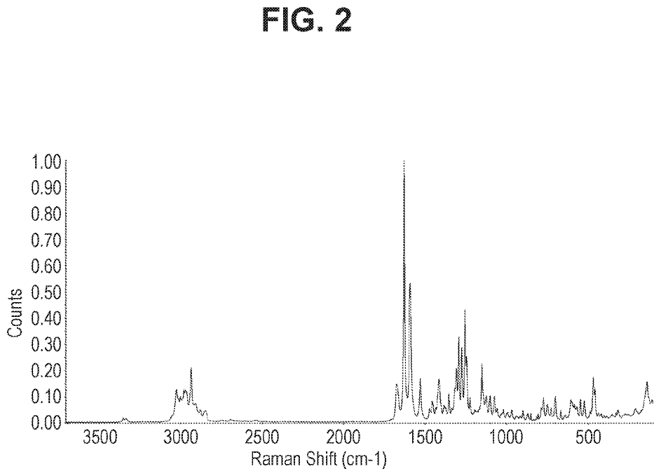

In specific embodiments, PF-06873600 free base (Form 1) has a Raman spectrum comprising: (a) one, two, three, four, five, or more than five wavenumber (cm⁻¹) values selected from the group consisting of the values in Table 2 in cm⁻¹±2 cm⁻¹; (b) one, two, three, four or five wavenumber (cm⁻¹) values selected from the group consisting of the characteristic values in Table 2 in cm⁻¹±2 cm⁻¹; or (c) wavenumber (cm⁻¹) values essentially the same as shown in FIG. 2.

In one embodiment, PF-06873600 free base (Form 1) has a ¹³C solid state NMR spectrum comprising one or more resonance (ppm) values selected from the group consisting of: 28.8, 42.0, 123.0, 133.2 and 161.4 ppm±0.2 ppm. In another embodiment, PF-06873600 free base (Form 1) has a ¹³C solid state NMR spectrum comprising two or more resonance (ppm) values selected from the group consisting of: 28.8, 42.0, 123.0, 133.2 and 161.4 ppm±0.2 ppm. In another embodiment, PF-06873600 free base (Form 1) has a ¹³C solid state NMR spectrum comprising three or more resonance (ppm) values selected from the group consisting of: 28.8, 42.0, 123.0, 133.2 and 161.4 ppm±0.2 ppm.

In other embodiments, PF-06873600 free base (Form 1) has a ¹³C solid state NMR spectrum comprising the resonance (ppm) values of: 28.8, 133.2 and 161.4 ppm±0.2 ppm. In some embodiments, PF-06873600 free base (Form 1) has a ¹³C solid state NMR spectrum comprising the resonance (ppm) value of: 28.8 ppm±0.2 ppm. In another embodiment, Form 1 has a ¹³C solid state NMR spectrum comprising the resonance (ppm) value of: 133.2 ppm±0.2 ppm. In another embodiment, Form 1 has a ¹³C solid state NMR spectrum comprising the resonance (ppm) value of: 161.4 ppm±0.2 ppm. In some such embodiments, Form 1 has a ¹³C solid state NMR spectrum further comprising the resonance (ppm) value of: 42.0 ppm±0.2 ppm. In other such embodiments, Form 1 has a ¹³C solid state NMR spectrum further comprising the resonance (ppm) value of: 123.0 ppm±0.2 ppm.

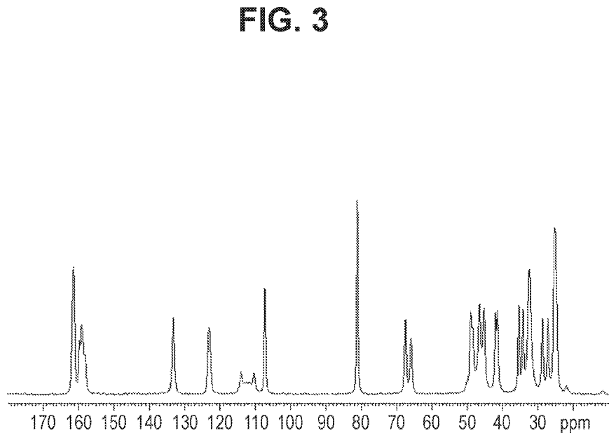

In specific embodiments, PF-06873600 free base (Form 1) has a ¹³C solid state NMR spectrum (ppm) comprising: (a) one, two, three, four, five, or more than five resonance (ppm) values selected from the group consisting of the values in Table 3 in ppm±0.2 ppm; (b) one, two, three, four or five resonance (ppm) values selected from the group consisting of the characteristic values in Table 3 in ppm±0.2 ppm; or (c) resonance (ppm) values essentially the same as shown in FIG. 3.

In one embodiment, PF-06873600 free base (Form 1) has a ¹⁹F solid state NMR spectrum comprising one or more resonance (ppm) values selected from the group consisting of: −109.6 and −122.7 ppm±0.2 ppm.

In another embodiment, PF-06873600 free base (Form 1) has a ¹⁹F solid state NMR spectrum comprising a resonance (ppm) value of: −109.6 ppm±0.2 ppm. In another embodiment, Form 1 has a ¹⁹F solid state NMR spectrum (ppm) comprising a resonance (ppm) value of: −122.7 ppm±0.2 ppm. In another embodiment, PF-06873600 free base (Form 1) has a ¹⁹F solid state NMR spectrum comprising resonance (ppm) values of: −109.6 and −122.7 ppm±0.2 ppm.

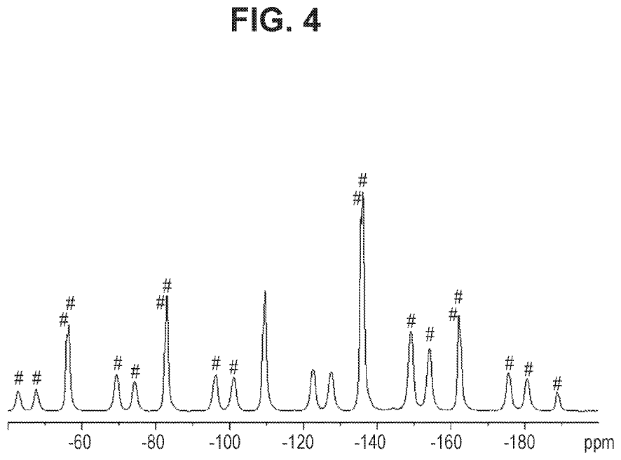

In another embodiment, Form 1 has a ¹⁹F solid state NMR spectrum (ppm) comprising: (a) one or two resonance (ppm) values selected from the group consisting of the values in Table 4 in ppm±0.2 ppm; or (b) resonance (ppm) values essentially the same as shown in FIG. 4.

In further embodiments, PF-06873600 free base (Form 1) is characterized by a combination of two, three or four of the embodiments described above that are not inconsistent with each other. Exemplary embodiments that may be used to uniquely characterize Form 1 of PF-06873600 free base are provided below.

In one embodiment, PF-06873600 free base (Form 1) has a powder X-ray diffraction pattern comprising peaks at 2θ values of: 9.6, 18.3 and 22.1 °2θ±0.2 °2θ.

In another embodiment, PF-06873600 free base (Form 1) has a powder X-ray diffraction pattern comprising peaks at 2θ values of: 6.9, 9.6, 18.3 and 22.1 °2θ±0.2 °2θ.

In another embodiment, PF-06873600 free base (Form 1) has: (a) a powder X-ray diffraction pattern comprising peaks at 2θ value of: 9.6, 18.3 and 22.1 °2θ±0.2 °2θ; and (b) a Raman spectrum comprising wavenumber (cm⁻¹) values of: 1589, 1626 and 1673 cm⁻¹±2 cm⁻¹.

In another embodiment, PF-06873600 free base (Form 1) has: (a) a powder X-ray diffraction pattern comprising peaks at 2θ values of: 9.6, 18.3 and 22.1 °2θ±0.2 °2θ; and (b) a ¹³C solid state NMR spectrum comprising resonance (ppm) values of: 28.8, 133.2 and 161.4 ppm±0.2 ppm.

In another embodiment, PF-06873600 free base (Form 1) has: (a) a powder X-ray diffraction pattern comprising peaks at 2θ values of: 9.6, 18.3 and 22.1 °2θ±0.2 °2θ; and (b) a ¹⁹F solid state NMR spectrum comprising a resonance (ppm) value of: −109.6 ppm±0.2 ppm.

In another embodiment, PF-06873600 free base (Form 1) has: (a) a powder X-ray diffraction pattern comprising peaks at 2θ value of: 9.6, 18.3 and 22.1 °2θ±0.2 °2θ; (b) a Raman spectrum comprising wavenumber (cm⁻¹) values of: 1589, 1626 and 1673 cm⁻¹±2 cm⁻¹; and (c) a ¹³C solid state NMR spectrum comprising resonance (ppm) values of: 28.8, 133.2 and 161.4 ppm±0.2 ppm.

In another embodiment, PF-06873600 free base (Form 1) has: (a) a powder X-ray diffraction pattern comprising peaks at 2θ values of: 9.6, 18.3 and 22.1 °2θ±0.2 °2θ; (b) a Raman spectrum comprising wavenumber (cm⁻¹) values of: 1589, 1626 and 1673 cm⁻¹±2 cm⁻¹; and (c) a ¹⁹F solid state NMR spectrum comprising a resonance (ppm) value of: −109.6 ppm±0.2 ppm.

In another embodiment, PF-06873600 free base (Form 1) has: (a) a powder X-ray diffraction pattern comprising peaks at 2θ values of: 9.6, 18.3 and 22.1 °2θ±0.2 °2θ; (b) a Raman spectrum comprising wavenumber (cm⁻¹) values of: 1589, 1626 and 1673 cm⁻¹±2 cm⁻¹; (c) a ¹³C solid state NMR spectrum comprising resonance (ppm) values of: 28.8, 133.2 and 161.4 ppm±0.2 ppm; and (d) a ¹⁹F solid state NMR spectrum comprising a resonance (ppm) value of: −109.6 ppm±0.2 ppm.

In another embodiment, PF-06873600 free base (Form 1) has: (a) a powder X-ray diffraction pattern comprising peaks at 2θ value of: 6.9, 9.6, 18.3 and 22.1 °2θ±0.2 °2θ; and (b) a Raman spectrum comprising wavenumber (cm⁻¹) values of: 1589, 1626 and 1673 cm⁻¹+2 cm⁻¹.

In another embodiment, PF-06873600 free base (Form 1) has: (a) a powder X-ray diffraction pattern comprising peaks at 2θ values of: 6.9, 9.6, 18.3 and 22.1 °2θ±0.2 °2θ; and (b) a ¹³C solid state NMR spectrum comprising resonance (ppm) values of: 28.8, 133.2 and 161.4 ppm±0.2 ppm.

In another embodiment, PF-06873600 free base (Form 1) has: (a) a powder X-ray diffraction pattern comprising peaks at 2θ values of: 6.9, 9.6, 18.3 and 22.1 °2θ±0.2 °2θ; and (b) a ¹⁹F solid state NMR spectrum comprising a resonance (ppm) value of: −109.6 ppm±0.2 ppm.

In another embodiment, PF-06873600 free base (Form 1) has: (a) a powder X-ray diffraction pattern comprising peaks at 2θ value of: 6.9, 9.6, 18.3 and 22.1 °2θ±0.2 °2θ; (b) a Raman spectrum comprising wavenumber (cm⁻¹) values of: 1589, 1626 and 1673 cm⁻¹±2 cm⁻¹; and (c) a ¹³C solid state NMR spectrum comprising resonance (ppm) values of: 28.8, 133.2 and 161.4 ppm±0.2 ppm.

In another embodiment, PF-06873600 free base (Form 1) has: (a) a powder X-ray diffraction pattern comprising peaks at 2θ values of: 6.9, 9.6, 18.3 and 22.1 °2θ±0.2 °2θ; (b) a Raman spectrum comprising wavenumber (cm⁻¹) values of: 1589, 1626 and 1673 cm⁻¹±2 cm⁻¹; and (c) a ¹⁹F solid state NMR spectrum comprising a resonance (ppm) value of: −109.6 ppm±0.2 ppm.

In another embodiment, PF-06873600 free base (Form 1) has: (a) a powder X-ray diffraction pattern comprising peaks at 2θ values of: 6.9, 9.6, 18.3 and 22.1 °2θ±0.2 °2θ; (b) a Raman spectrum comprising wavenumber (cm⁻¹) values of: 1589, 1626 and 1673 cm⁻¹±2 cm⁻¹; (c) a ¹³C solid state NMR spectrum comprising resonance (ppm) values of: 28.8, 133.2 and 161.4 ppm±0.2 ppm; and (d) a ¹⁹F solid state NMR spectrum comprising a resonance (ppm) value of: −109.6 ppm±0.2 ppm.

In another embodiment, PF-06873600 free base (Form 1) has a ¹⁹F solid state NMR spectrum comprising resonance (ppm) values of: −109.6 and −122.7 ppm±0.2 ppm.

In another embodiment, PF-06873600 free base (Form 1) has: (a) a ¹⁹F solid state NMR spectrum comprising resonance (ppm) values of: −109.6 and −122.7 ppm±0.2 ppm; and (b) a powder X-ray diffraction pattern comprising peaks at 2θ value of: 9.6, 18.3 and 22.1 °2θ±0.2 °2θ.

In another embodiment, PF-06873600 free base (Form 1) has: (a) a ¹⁹F solid state NMR spectrum comprising resonance (ppm) values of: −109.6 and −122.7 ppm±0.2 ppm; and (b) a Raman spectrum comprising wavenumber (cm⁻¹) values of: 1589, 1626 and 1673 cm⁻¹±2 cm⁻¹.

In another embodiment, PF-06873600 free base (Form 1) has: (a) a ¹⁹F solid state NMR spectrum comprising resonance (ppm) values of: −109.6 and −122.7 ppm±0.2 ppm; and (b) a ¹³C solid state NMR spectrum comprising resonance (ppm) values of: 28.8, 133.2 and 161.4 ppm±0.2 ppm.

In another embodiment, PF-06873600 free base (Form 1) has: (a) a ¹⁹F solid state NMR spectrum comprising resonance (ppm) values of: −109.6 and −122.7 ppm±0.2 ppm; (b) a powder X-ray diffraction pattern comprising peaks at 2θ value of: 9.6, 18.3 and 22.1 °2θ±0.2 °2θ; and (c) a Raman spectrum comprising wavenumber (cm⁻¹) values of: 1589, 1626 and 1673 cm⁻¹±2 cm⁻¹.

In another embodiment, PF-06873600 free base (Form 1) has: (a) a ¹⁹F solid state NMR spectrum comprising resonance (ppm) values of: −109.6 and −122.7 ppm±0.2 ppm; (b) a powder X-ray diffraction pattern comprising peaks at 2θ value of: 9.6, 18.3 and 22.1 °2θ±0.2 °2θ; and (c) a ¹³C solid state NMR spectrum comprising resonance (ppm) values of: 28.8, 133.2 and 161.4 ppm±0.2 ppm.

In another embodiment, PF-06873600 free base (Form 1) has: (a) a ¹⁹F solid state NMR spectrum comprising resonance (ppm) values of: −109.6 and −122.7 ppm±0.2 ppm; (b) a powder X-ray diffraction pattern comprising peaks at 2θ value of: 9.6, 18.3 and 22.1 °2θ±0.2 °2θ; (c) a Raman spectrum comprising wavenumber (cm⁻¹) values of: 1589, 1626 and 1673 cm⁻¹±2 cm⁻¹; and (d) a ¹³C solid state NMR spectrum comprising resonance (ppm) values of: 28.8, 133.2 and 161.4 ppm±0.2 ppm.

In another embodiment, PF-06873600 free base (Form 1) has a Raman spectrum comprising wavenumber (cm⁻¹) values of: 1254, 1528, 1589, 1626 and 1673 cm⁻¹±2 cm¹.

In another embodiment, PF-06873600 free base (Form 1) has a ¹³C solid state NMR spectrum comprising resonance (ppm) values of: 28.8, 42.0, 123.0, 133.2 and 161.4 ppm±0.2 ppm.

In another aspect, the invention provides PF-06873600 free base (Form 1), having: (a) a powder X-ray diffraction (PXRD) pattern comprising peaks at 2θ values of: 9.6, 18.3 and 22.1 °2θ±0.2 °2θ; (b) a Raman spectrum comprising one or more wavenumber (cm⁻¹) values selected from the group consisting of: 1589, 1626 and 1673 cm⁻¹±2 cm⁻¹; (c) a ¹³C solid state NMR spectrum comprising one or more resonance (ppm) values selected from the group consisting of: 28.8, 133.2 and 161.4 ppm±0.2 ppm; or (d) a ¹⁹F solid state NMR spectrum comprising a resonance (ppm) value of: −109.6 ppm±0.2 ppm; or a combination of two or more of (a), (b), (c) and (d). Each of the embodiments described herein for individual methods of characterization may be combined with or further limit this aspect, provided the embodiments are not inconsistent with each other.

In another aspect, the invention provides a pharmaceutical composition comprising the crystalline form of PF-06873600 free base (Form 1) according to any of the embodiments described herein, and a pharmaceutically acceptable carrier or excipient.

In another aspect, the invention provides method of treating abnormal cell growth in a mammal, preferably a human, comprising administering to the mammal a therapeutically effective amount of the crystalline form of PF-06873600 free base (Form 1) according to any of the embodiments described herein.

In another aspect, the invention provides method of treating abnormal cell growth in a mammal, preferably a human, comprising administering to the mammal a therapeutically effective amount of a pharmaceutical composition comprising the crystalline form of PF-06873600 free base (Form 1) according to any of the embodiments described herein.

In another aspect, the invention the crystalline form of PF-06873600 free base (Form 1) according to any of the embodiments described herein for use in treating abnormal cell growth in a mammal, preferably a human.

In another aspect, the invention provides the use of the crystalline form of PF-06873600 free base (Form 1) according to any of the embodiments described herein in treating abnormal cell growth in a mammal, preferably a human.

In another aspect, the invention provides use of the crystalline form of PF-06873600 free base (Form 1) according to any of the embodiments described herein in the manufacture of a medicament for use in a treating abnormal cell growth in a mammal, preferably a human.

In frequent embodiments of the methods, compositions and uses described herein, the abnormal cell growth is cancer.

The term “therapeutically effective amount” as used herein refers to that amount of a compound being administered which will relieve to some extent one or more of the symptoms of the disorder being treated. In reference to the treatment of cancer, a therapeutically effective amount refers to that amount which has the effect of (1) reducing the size of the tumor, (2) inhibiting (that is, slowing to some extent, preferably stopping) tumor metastasis, (3) inhibiting to some extent (that is, slowing to some extent, preferably stopping) tumor growth or tumor invasiveness, and/or (4) relieving to some extent (or, preferably, eliminating) one or more signs or symptoms associated with the cancer.

As used herein, “mammal” refers to a human or animal subject. In certain preferred embodiments, the mammal is a human.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above. The term “treating” also includes adjuvant and neo-adjuvant treatment of a subject.

“Abnormal cell growth”, as used herein, unless otherwise indicated, refers to cell growth that is independent of normal regulatory mechanisms (e.g., loss of contact inhibition). Abnormal cell growth may be benign (not cancerous), or malignant (cancerous). In frequent embodiments of the methods provided herein, the abnormal cell growth is cancer.

As used herein “cancer” refers to any malignant and/or invasive growth or tumor caused by abnormal cell growth. The term “cancer” includes but is not limited to a primary cancer that originates at a specific site in the body, a metastatic cancer that has spread from the place in which it started to other parts of the body, a recurrence from the original primary cancer after remission, and a second primary cancer that is a new primary cancer in a person with a history of previous cancer of different type from latter one.

Pharmaceutical compositions of the present invention may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

Exemplary parenteral administration forms include solutions or suspensions of active compounds in sterile aqueous solutions, for example, aqueous propylene glycol or dextrose solutions. Such dosage forms can be suitably buffered, if desired.

Suitable pharmaceutical carriers include inert diluents or fillers, water and various organic solvents. The pharmaceutical compositions may, if desired, contain additional ingredients such as flavorings, binders, excipients and the like. Thus, for oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Preferred materials include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

Methods of preparing various pharmaceutical compositions with a specific amount of active compound are known, or will be apparent, to those skilled in this art. For examples, see Remington's Pharmaceutical Sciences, Mack Publishing Company, Easter, Pa., 15th Edition (1975).

### EXAMPLES

The examples and preparations provided below further illustrate and exemplify particular aspects and embodiments of the invention. It is to be understood that the scope of the present invention is not limited by the scope of the following examples.

### General Method 1. Powder X-Ray Diffraction (PXRD)

Instrument Method:

Powder X-ray diffraction analysis was conducted using a Bruker AXS D4 Endeavor diffractometer equipped with a Cu radiation source. The divergence slit was set at 0.6 mm while the secondary optics used variable slits. Diffracted radiation was detected by a PSD-Lynx Eye detector. The X-ray tube voltage and amperage were set to 40 kV and 40 mA respectively. Data was collected in the Theta-2Theta goniometer at the Cu wavelength (CuK_{α̅}=1.5418 λ) from 3.0 to 40.0 degrees 2-Theta using a step size of 0.0185 degrees and a step time of 5 second. Note that Cu K-beta wavelength was filtered. Samples were prepared by placing them in a silicon low background sample holder and rotated during collection. Data were collected using Bruker DIFFRAC Plus software and analysis was performed by EVA diffract plus software.

Peak Picking Method:

The PXRD data file was not processed prior to peak searching. Using the peak search algorithm in the EVA software, peaks selected with a threshold value of 1 were used to make preliminary peak assignments. To ensure validity, adjustments were manually made; the output of automated assignments was visually checked, and peak positions were adjusted to the peak maximum. Peaks with relative intensity of ≥3% were generally chosen. Typically, the peaks which were not resolved or were consistent with noise were not selected. A typical error associated with the peak position from PXRD stated in USP up to +/−0.2° 2-Theta (USP-941).

### General Method 2. Raman Spectroscopy

Instrument Method:

Raman spectra were collected using a Nicolet NXR FT-Raman accessory attached to the FT-IR bench. The spectrometer is equipped with a 1064 nm Nd:YVO₄ laser and a liquid nitrogen cooled Germanium detector. Prior to data acquisition, instrument performance and calibration verifications were conducted using polystyrene. API samples were analyzed in glass NMR tubes that were static during spectral collection. The spectra were collected using 0.5 W of laser power and 512 co-added scans. The collection range was 3700-100 cm⁻¹. These spectra were recorded using 2 cm⁻¹ resolution and Happ-Genzel apodization. Utilizing the Raman method above, the possible variability associated with a spectral measurement is ±2 cm⁻¹.

Peak Picking Method:

The intensity scale was normalized to 1 prior to peak picking. Peaks were manually identified using the Thermo Nicolet Omnic 9.7.46 software. Peak position was picked at the peak maximum, and peaks were only identified as such, if there was a slope on each side; shoulders on peaks were not included. For neat Form 1 API an absolute threshold of 0.006 with a sensitivity of 84 was utilized during peak picking. The peak position has been rounded to the nearest whole number using standard practice (0.5 rounds up, 0.4 rounds down). Peaks with normalized peak intensity between (1-0.75), (0.74-0.30), (0.29-0) were labeled as strong, medium and weak, respectively. The relative peak intensity values are also illustrated in this report.

The characteristic peaks for these forms were chosen based on their intensity, as well as peak position.

General Method 3. Solid State NMR (ssNMR) Spectroscopy:

Instrument Method:

Solid state NMR (ssNMR) analysis was conducted on a CPMAS probe positioned into a Bruker-BioSpin Avance III 500 MHz (¹H frequency) NMR spectrometer. Material was packed into a 4 mm rotor sealed with a standard drive cap. The ¹³C ssNMR spectrum was collected using a proton decoupled cross-polarization magic angle spinning (CPMAS) experiment using a magic angle spinning rate of 14.0 kHz. The cross-polarization contact time was set to 2 ms and the recycle delay to 5 seconds. A phase modulated proton decoupling field of 80-90 kHz was applied during spectral acquisition. The number of scans was adjusted to obtain an adequate signal to noise ratio; 1024 scans were collected for API sample and 8192 scans collected for drug product samples. The ¹³C chemical shift scale was referenced using a ¹³C CPMAS experiment on an external standard of crystalline adamantane, setting its up-field resonance to 29.5 ppm (as determined from neat TMS). The ¹⁹F ssNMR spectrum was collected using a proton decoupled magic angle spinning (MAS) experiment using a magic angle spinning rate of 12.5 kHz. A phase modulated proton decoupling field of 80-90 kHz was applied during spectral acquisition. 256 scans were collected with a recycle delay of 40 seconds. The ¹⁹F chemical shift scale was referenced using a ¹⁹F MAS experiment on an external standard of trifluoroacetic acid and water (50/50 volume/volume), setting its resonance to −76.54 ppm (as determined from neat TMS).

Peak Picking Method:

Automatic peak picking was performed using Bruker-BioSpin TopSpin version 3.5 software. Generally, a threshold value of 3% relative intensity was used for preliminary peak selection. The output of the automated peak picking was visually checked to ensure validity and adjustments were manually made if necessary.

Although specific solid-state NMR peak values are reported herein there does exist a range for these peak values due to differences in instruments, samples, and sample preparation. This is common practice in the art of solid-state NMR because of the variation inherent in peak positions. A typical variability for a ¹³C and ¹⁹F chemical shift x-axis value is on the order of plus or minus 0.2 ppm for a crystalline solid. The solid-state NMR peak heights reported herein are relative intensities. Solid state NMR intensities can vary depending on the actual setup of the CPMAS experimental parameters and the thermal history of the sample.

### General Synthetic Methods:

The PF-06873600 free base starting material can be prepared as described in Example 10 of U.S. Pat. No. 10,233,188. The intermediates identified as Compound 1, Compound 2 and Compound 3 can be prepared according to Example 2 of U.S. Pat. No. 10,233,188. PF-06873600 free base, Form 1 was initially prepared on lab-scale as described in Example 1 herein. PF-06873600 free base (Form 1) crystals prepared as described in Example 1 may be used as seed crystals for larger scale experiments. Seeding is frequently used to initiate crystallization at the desired supersaturation level and improve batch consistency but is not typically required to obtain crystalline material.

### Example 1

### Lab-Scale Preparation of 6-(difluoromethyl)-8-[(1R,2R)-2-hydroxy-2-methylcyclopentyl]-2-{[1-(methylsulfonyl)piperidin-4-yl]amino}pyrido[2,3-d]pyrimidin-7(8H)-one (PF-06873600) Anhydrous Free Base (Form 1)

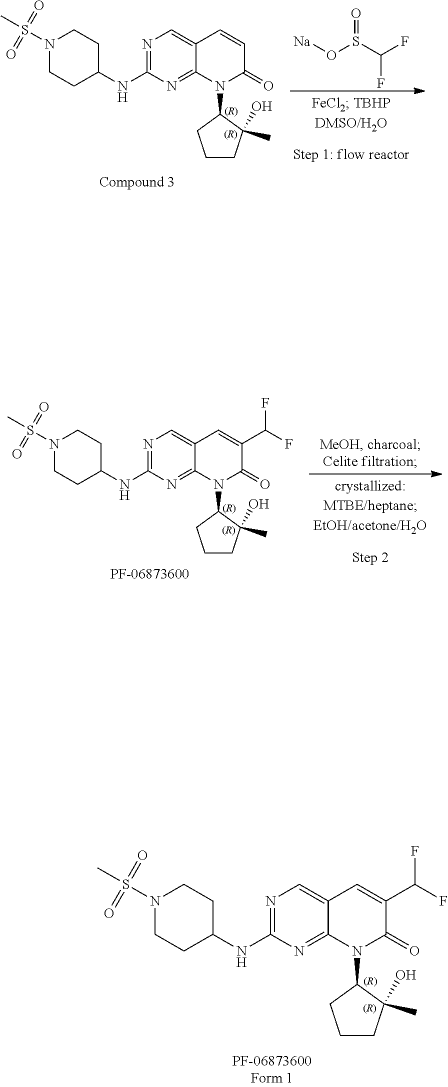

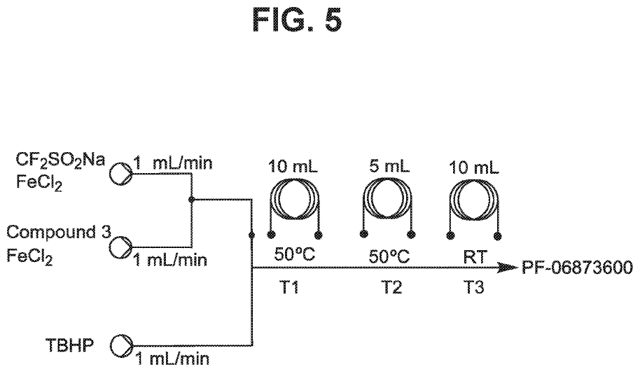

Step 1: The following solutions were prepared for use in a flow reactor (configured as shown in FIG. 5 and generally as in Example 133/134 of U.S. Pat. No. 10,233,188): Bottle 1, sodium difluoromethanesulfinate (4910 mg, 35.6 mmol) and iron(II) chloride (118 mg, 0.593 mmol) in 9/1 DMSO/water (135 mL DMSO/15 mL water); Bottle 2, tent-butyl hydroperoxide (TBHP, 3210 mg, 35.6 mmol, 3000 μL) in 150 mL of DMSO; Bottle 3, Compound 3 (prepared as described in Example 2 of U.S. Pat. No. 10,233,188), and iron(II) chloride (118 mg, 0.593 mmol) in 100 mL of DMSO.

The solutions were passed through the flow reactor at a rate of 1 mL/min. The temperature at T1 and T2 was 50° C. and at T3 was at room temperature. After the substrate solution was consumed, the product mixture was poured into ice/10% aqueous sodium ethylenediaminetetraacetic acid (EDTA) (13500 g, 35.6 mmol) and vigorously stirred for 10 min. The aqueous solution was extracted with ethyl acetate (4×300 mL) and the organic layers were combined, washed with saturated sodium bicarbonate (300 mL) and brine (300×2 mL), dried over sodium sulfate and concentrated. The residue was loaded onto a silica column and eluted with ethyl acetate/heptane 0-100%. 1350 mg of PF-06873600 was obtained (48.3% yield).

Step 2: PF-06873600 (2.35 g, 4.863 mmol) prepared in two batches according to Step 1, was dissolved in methanol (300 mL). Activated charcoal (20 g, 1700 mmol) was added and the slurry was stirred for 2 hours. The charcoal was removed by filtration through a bed of CELITE® on a glass fiber filter. The filter cake was washed with methanol and acetone and the volatiles were removed. The residual PF-06873600 (foam) was crystallized from a mixture of methyl tert-butyl ether (MTBE) and heptane, followed by a second crystallization in ethanol/acetone/water to give PF-06873600 as a white crystalline solid (small needles).

¹H NMR (400 MHz, DMSO-d6, 80° C.) Shift 8.74 (s, 1H), 8.04 (s, 1H), 7.77 (dd, J=5.93, 11.19 Hz, 1H), 6.60-7.00 (m, 1H), 5.75-5.95 (m, 1H), 4.08 (s, 1H), 3.91-4.07 (m, J=6.48 Hz, 1H), 3.63 (t, J=11.62 Hz, 2H), 2.90-2.98 (m, 2H), 2.88 (s, 3H), 2.16-2.26 (m, 1H), 2.06-2.16 (m, 1H), 1.97-2.06 (m, J=12.23 Hz, 2H), 1.85-1.95 (m, J=4.28 Hz, 2H), 1.68-1.79 (m, 2H), 1.50-1.68 (m, 1H), 1.04 (s, 3H); ¹⁹F NMR (377 MHz, DMSO-d6) Shift −122.82-111.49 (m, 2F); Optical rotation: [α]_{D}²²−24.4 (c 0.5, CHCl₃).

### Example 2

### Large Scale Preparation of 6-(difluoromethyl)-8-[(1R,2R)-2-hydroxy-2-methylcyclopentyl]-2-{[1-(methylsulfonyl)piperidin-4-yl]amino}pyrido[2,3-D]pyrimidin-7(8H)-one (PF-06873600) Anhydrous Free Base (Form 1)

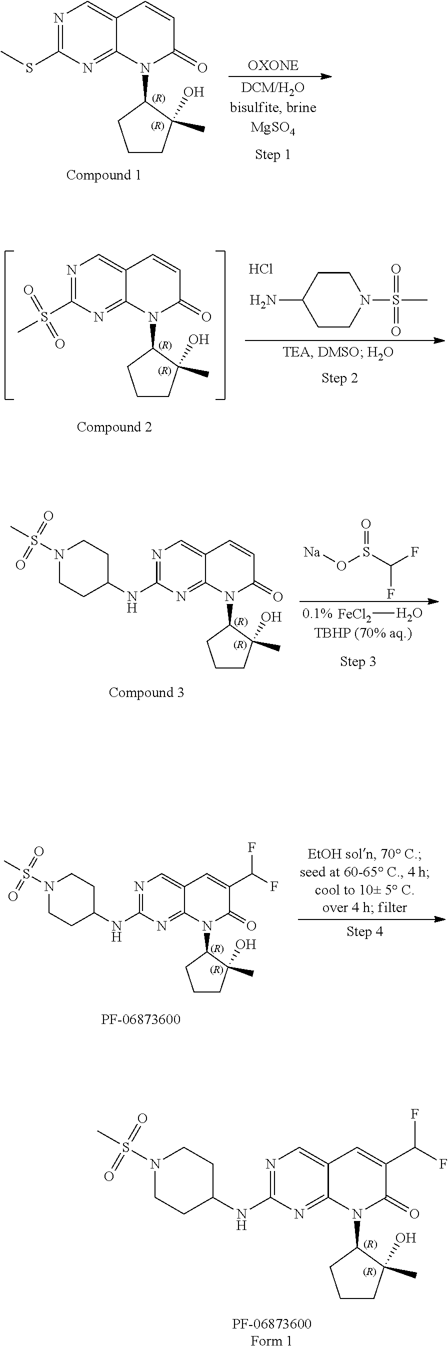

Step 1: 8-[(1R,2R)-2-hydroxy-2-methylcyclopentyl]-2-(methylsulfanyl)pyrido[2,3-d]pyrimidin-7(8H)-one (Compound 1) (7.0 kg) was dissolved in 69 L dichloromethane (DCM) in a 100 L reactor at 20±5° C.

A 200 L reactor was charged with 70 L water and 20.8 kg OXONE® (CAS no. 37222-66-5) at 20±5° C. and mixed for 5 min to provide a thin slurry. The OXONE® mixture was cooled to 0±5° C. The solution of Compound 1 was added to the reactor over 15 minutes while maintaining a temperature of 0±10° C. The reactor was warmed to 20±5° C. and held for 3 hours.

Once the reaction was complete by ultra-high-pressure liquid chromatography (UPLC), the mixture was cooled to 0±5° C. and quenched by addition of aqueous sodium bisulfite (11.75 kg anhydrous NaHSO₃ in 28 L H₂O) at 0-10° C. over 15 minutes. The mixture was warmed to 20±5° C. The layers were separated, and the organic layer was washed with dilute brine (5.04 kg NaCl dissolved in 33.6L water, 13% aq.) and dried by slurrying with 2 kg anhydrous magnesium sulfate at 20±5° C. for 15 min. The drying agent was removed by filtration through a Nutsche filter and the filter cake was washed with 7 L DCM and blown dry under a stream of nitrogen over 30 min.

The filtrate was transferred to a 100 L reactor and concentrated under vacuum at 20±15° C. to a volume of 14-16 L. To purge the DCM, 13 L of DMSO was added and the solution was concentrated under vacuum at 20±15° C. to a volume of 20-26 L. The temperature was adjusted to 20±5° C. Compound 2 was taken into Step 2 without further isolation.

Step 2: A 200 L reactor was charged with 56 L of DMSO and 4.62 kg of triethylamine (TEA) was added. The reactor was swept with nitrogen and 9.7 kg of 4-amino-1-methanesulfonylpiperidine hydrochloride (CAS no. 651057-01-1) was added under nitrogen at 20±5° C. and held at 20±5° C. for 30 min. The DMSO solution of Compound 2 to the 200 L reactor at 20±5° C. The mixture was heated to 25±5° C. and stirred for 18 hours. Once the reaction was complete by UPLC, the reaction mixture was heated to 45±5° C. and diluted with 70 L of water at 45±5° C. The solution was seeded with 0.018 kg of seed crystals and the mixture held at 45±5° C. for 1 hour. Additional water (26 L) was added and the mixture was cooled slowly to 15±5° C. over 5 hours and held for 1.5 hours.

The solid was collected by filtration through a Nutsche filter. The filter cake was washed with 21 L of water and dried under nitrogen for 1 hour, then dried in a vacuum oven at 50±5° C. for 4 hours to give Compound 3 as a white solid, consistent with material prepared in Example 2 of U.S. Pat. No. 10,233,188.

Step 3: A 100 L reactor was charged with 9 L of DMSO. Luperox TBH70X tert-butyl hydroperoxide 70 wt % in water was added at 15±5° C. and held at that temperature until homogenous. A 200 L reactor was charged with 9 L water and 46 L of DMSO at 20±10° C. Sodium difluoromethanesulfinate (5.41 kg) (CAS No. 275818-95-6) was added and the mixture was held at 20±10° C. for 10 minutes. 7 L of DMSO was added while maintaining the temperature at 20±5° C. Compound 3 (6.6 kg) was added followed by iron (II) chloride tetrahydrate (0.318 kg). 12 L of DMSO was added to the reactor, which was then cooled to 5±5° C.

The tert-butyl hydroperoxide mixture was transferred to the 200 L reactor at 0 to 10° C. over 1 hour and held at 5±5° C. for 15 minutes. The reaction was monitored by UPLC. Following completion, the reaction mixture was diluted with 40 L of water at 15 to 20° C. The reaction mixture was partitioned between water and 33 L of ethyl acetate (EtOAc), the aqueous layer was extracted with two 33 L portions of EtOAc, and the combined EtOAc layers were washed with aqueous sodium bisulfite solution (2.44 kg sodium bisulfite anhydrous in 13 L of water), followed by 13 L of water. The washed organic solution was concentrated under vacuum at 35±15° C. to provide a viscous oil. The solvent was exchanged to acetonitrile (CH₃CN) by addition of 23 L of CH₃CN and mixing to dissolve the oil, then concentrating under vacuum at 35±15° C. to provide an oil (10-11 L volume). Toluene (9.6 kg) was added to the crude concentrate and the resulting CH₃CN/toluene solution of PF-06873600 was purified by chromatography on silica gel-flash.

Step 4: PF-06873600 (3.8 kg) prepared according to Step 3 and ethanol (104 L, ˜37 g/L) were added to a reactor and heated to dissolve at 70±10° C. The temperature was adjusted to 60-65° C. and ˜250 mL of seed slurry (prepared as described below) was added. The reactor was held at 60-65° C. for 4 hours and then cooled to 10±5° C. over 4 hours and held for 1 hour. The reactor was then heated to 55±5° C. over 30 minutes and concentrated at 40±10° C. for about 2 hours. The concentrated slurry was cooled to 10±5° C. over 3 hours and held for 1 hour. The resulting slurry was then isolated via filtration. The filter cake was washed with ethanol and dried in a vacuum oven at 40±5° C.

The seed slurry was separately prepared by mixing PF-06873600 (0.132 kg) and ethanol (0.5 L, ˜264 g/L) in a flask at 20±5° C. for 30 minutes to provide a uniform slurry.

### Example 3

### Alternative Preparation of PF-06873600 Anhydrous Free Base (Form 1)

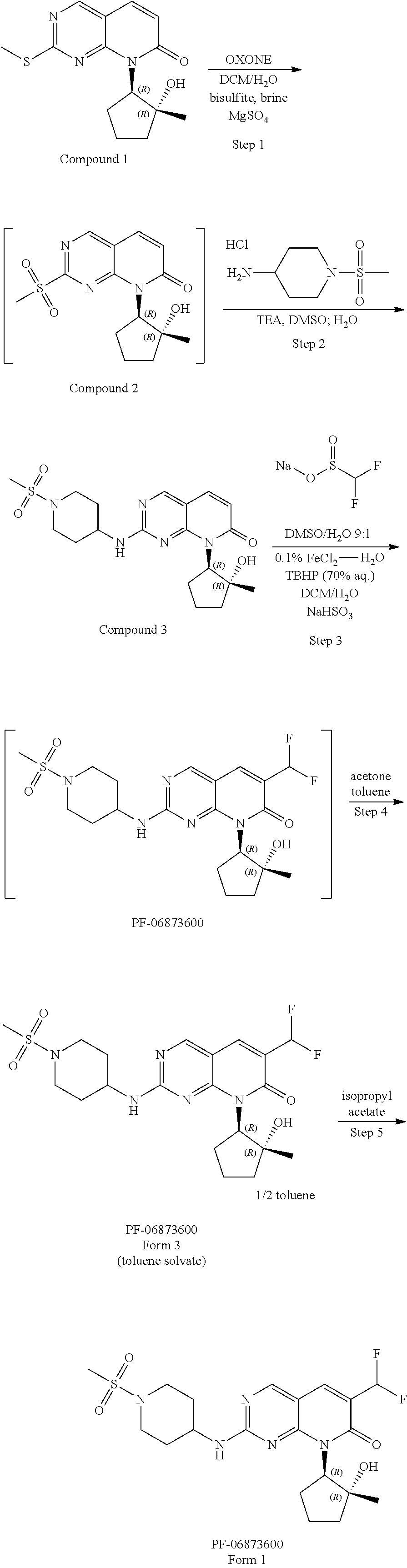

Steps 1-3 were conducted as described in Example 2. The intermediate PF-06873600 was converted to the toluene solvate (Form 3) by dissolution in hot acetone and cooling to ambient temperature, then diluting with ten-fold toluene and allowing to crystallize. The resulting slurry of Form 3 was collected by filtration and washed with toluene. The Form 3 solvate was dried with suction under a stream of nitrogen. The PF-06873600 toluene solvate (Form 3) (7.1 kg) and isopropyl acetate (179L, ˜83 g/L) were added to a reactor. The mixture was then heated to 80-85° C. over 1 hour and held for 8 hours, followed by cooling to 5-10° C. over 6 hours. The mixture was held at this temperature for 4 hours and the resulting slurry was then isolated via filtration to provide PF-06873600 Form 1, consistent with authentic material prepared in Example 1 and 2.

### Example 4

### Characterization of PF-06873600 Anhydrous Free Base (Form 1)

PXRD Data

FIG. 1 shows PXRD data for PF-06873600 free base (Form 1), collected according to General Method 1. A list of PXRD peaks at diffraction angles 2-Theta ° (°2θ)±0.2 °2θ and their relative intensities is provided in Table 1.

TABLE 1
PXRD Peak List for PF-06873600 free base Form 1 (2-Theta °)

| | Relative |
|---|---|
| Angle (2-theta °) ± 0.2 °2θ | Intensity (%) |
| 6.9 | 6.9 |
| 9.6 | 58.4 |
| 13.7 | 8.2 |
| 14.3 | 3.6 |
| 15.3 | 25.9 |
| 15.5 | 20.4 |
| 16.9 | 50.8 |
| 17.3 | 34.7 |
| 18.3 | 39.6 |
| 19.2 | 100.0 |
| 19.4 | 30.0 |
| 19.9 | 11.7 |
| 20.6 | 4.1 |
| 21.8 | 33.1 |
| 22.1 | 68.6 |
| 22.9 | 7.7 |
| 24.0 | 6.1 |
| 24.2 | 5.9 |
| 24.5 | 24.4 |
| 24.8 | 17.3 |
| 26.8 | 18.0 |
| 27.7 | 7.7 |
| 27.9 | 21.7 |
| 27.1 | 5.2 |
| 28.6 | 9.3 |
| 29.4 | 9.9 |
| 30.8 | 9.0 |
| 34.4 | 5.7 |
| 34.6 | 6.6 |
| 35.4 | 3.2 |
| 37.0 | 9.2 |
| 39.1 | 5.5 |

FT-Raman Data

FIG. 2 shows the FT-Raman spectrum of PF-06873600 free base (Form 1), collected according to General Method 2. A full list of FT-Raman peaks (cm⁻¹) and qualitative intensities is provided in Table 2 in cm⁻¹±2 cm⁻¹. Normalized peak intensities are indicated as follows: W=weak; M=medium; S=strong.

TABLE 2
FT Raman Peak List for PF-06873600 free base Form 1 (cm⁻¹)

| Peak position | Normalized | |
|---|---|---|
| cm⁻¹ ± 2 cm⁻¹ | intensity | Classification |
| 106 | 0.09 | W |
| 137 | 0.16 | W |
| 208 | 0.05 | W |
| 270 | 0.03 | W |
| 314 | 0.05 | W |
| 326 | 0.04 | W |
| 349 | 0.03 | W |
| 400 | 0.03 | W |
| 419 | 0.03 | W |
| 434 | 0.04 | W |
| 456 | 0.13 | W |
| 467 | 0.17 | W |
| 486 | 0.04 | W |
| 521 | 0.08 | W |
| 544 | 0.09 | W |
| 568 | 0.06 | W |
| 582 | 0.07 | W |
| 602 | 0.09 | W |
| 636 | 0.03 | W |
| 664 | 0.05 | W |
| 698 | 0.10 | W |
| 726 | 0.06 | W |
| 750 | 0.07 | W |
| 772 | 0.09 | W |
| 786 | 0.06 | W |
| 805 | 0.03 | W |
| 815 | 0.02 | W |
| 850 | 0.03 | W |
| 867 | 0.03 | W |
| 897 | 0.04 | W |
| 912 | 0.02 | W |
| 939 | 0.02 | W |
| 966 | 0.05 | W |
| 991 | 0.04 | W |
| 1017 | 0.05 | W |
| 1055 | 0.05 | W |
| 1073 | 0.10 | W |
| 1099 | 0.10 | W |
| 1124 | 0.10 | W |
| 1150 | 0.23 | W |
| 1193 | 0.05 | W |
| 1222 | 0.09 | W |
| 1244 | 0.25 | W |
| 1254 | 0.43 | M |
| 1272 | 0.29 | W |
| 1291 | 0.33 | M |
| 1305 | 0.21 | W |
| 1334 | 0.05 | W |
| 1351 | 0.11 | W |
| 1371 | 0.06 | W |
| 1379 | 0.07 | W |
| 1411 | 0.17 | W |
| 1433 | 0.06 | W |
| 1455 | 0.08 | W |
| 1472 | 0.05 | W |
| 1528 | 0.17 | W |
| 1589 | 0.53 | M |
| 1626 | 1.01 | S |
| 1673 | 0.15 | W |
| 2688 | 0.01 | W |
| 2848 | 0.04 | W |
| 2878 | 0.05 | W |
| 2909 | 0.07 | W |
| 2933 | 0.21 | W |
| 2967 | 0.12 | W |
| 2977 | 0.13 | W |
| 2998 | 0.10 | W |
| 3023 | 0.13 | W |
| 3350 | 0.02 | W |

ssNMR Data

FIG. 3 shows the carbon CPMAS spectrum of PF-06873600 free base (Form 1), which was collected according to General Method 3. Chemical shifts are expressed in parts per million (ppm) and are referenced to external sample of solid phase adamantane at 29.5 ppm. A list of ssNMR ¹³C chemical shifts (ppm) for Form 1 is provided in Table 3 in ppm±0.2 ppm.

TABLE 3
ssNMR ¹³C Chemical Shifts for PF-06873600
free base Form 1 (ppm)

| ¹³C Chemical Shifts | Relative |
|---|---|
| ppm ± 0.2 ppm | Intensity |
| 25.3 | 86 |
| 27.2 | 39 |
| 28.8 | 39 |
| 32.5 | 64 |
| 34.3 | 44 |
| 35.5 | 46 |
| 41.5 | 43 |
| 42.0 | 42 |
| 45.3 | 44 |
| 46.6 | 46 |
| 48.6 | 35 |
| 49.1 | 42 |
| 66.0 | 29 |
| 67.5 | 38 |
| 81.1 | 100 |
| 107.3 | 55 |
| 110.3 | 11 |
| 114.0 | 11 |
| 123.0 | 34 |
| 133.2 | 40 |
| 158.2 | 21 |
| 159.0 | 36 |
| 159.6 | 28 |
| 161.4 | 65 |

FIG. 4 shows the ¹⁹F fluorine MAS (ssNMR) spectrum of PF-06873600 free base (Form 1), collected according to General Method 3. Chemical shifts are expressed in parts per million (ppm) referenced to an external sample of trifluoroacetic acid (50% V/V in H₂O) at −76.54 ppm.

The ssNMR ¹⁹F chemical shift (ppm) for Form 1 is provided in Table 4 in ppm±0.2 ppm.

TABLE 4
ssNMR ¹⁹F Chemical Shifts for PF-06873600
free base Form 1 (ppm)

| ¹⁹F Chemical Shifts | |
|---|---|
| ppm ± 0.2 ppm | Intensity |
| −109.1 | 38 |
| −109.6 | 55 |
| −122.7 | 20 |
| −127.6 | 18 |

### Example 5

### Solid-State Chemical Stability of PF-06873600 Anhydrous Free Base (Form 1)

The chemical stability of PF-06873600 anhydrous free base (Form 1) was investigated at long term storage conditions (25° C./60% RH) for an extended time period and under accelerated stability conditions (40° C./75% RH) for a shorter period. Stability testing was conducted at 25° C./60% RH for 24 months (long term conditions) and at 40° C./75% RH for 6 months (accelerated conditions) and evaluated for appearance and purity by HPLC.

A photostability study was run under ICH conditions using light source option 2 ICH conditions. A change in appearance from an off-white powder to a pale-colored powder was observed for samples directly exposed to UV/Fluorescent conditions.

Stability samples of drug substance were packaged in double low-density polyethylene (LDPE) bags and desiccant within high-density polyethylene (HDPE) drum. Photostability samples were stored in a quartz Petri dish with a quartz lid.

TABLE 5
Chemical stability of PF-06873600 free base (Form 1)

| | | | Total impurities | | | | |
|---|---|---|---|---|---|---|---|
| | | Solid | Ini- | 3 | 6 | 12 | 24 |
| Conditions | Time | Form | tial | mos. | mos. | mos. | mos. |
| 25° C./60% RH | 24 mos. | Form 1 | 0.50 | 0.49 | 0.48 | 0.51 | 0.59 |
| 40° C./75% RH | 6 mos. | Form 1 | 0.50 | 0.47 | 0.47 | | |

TABLE 6
Photostability of PF-06873600 free base (Form 1)

| | | | Total impurities | |
|---|---|---|---|---|
| Conditions | Time | Solid Form | Initial | 8 days |
| ICH photostability, | 8 days | Form 1 | 0.58 | 0.64 |
| light source option 2 | | | | |

### Example 6

### Moisture Sorption of PF-06873600 Anhydrous Free Base (Form 1)

Water sorption and desorption studies were conducted on automated vapor sorption analyzer (TA instruments Q5000 SA). The microbalance was calibrated using a 100 mg standard weight. The relative humidity sensor was calibrated at 5.0, 11.3, 32.8, 52.8, 75.3, and 84.3% RH (25° C.) using saturated salt solutions. Approximately 30 mg of the powder sample was placed in the quartz sample holder and dried at ≤3% relative humidity (RH) at 25° C. The attainment of equilibrium was assumed when the weight change of the sample was <0.001 wt % in 5 min or by a maximum equilibration time of 120 minutes. The RH was then progressively increased to 90% in increments of 10% followed by a decrease to a final RH of 10% in 10% RH increments. The attainment of equilibrium was again assumed when the weight change of the sample was <0.001 wt % in 5 min or by a maximum equilibration time of 120 minutes. The weight gain at each of the % RH steps is based on the weight after the initial drying step. The data were analyzed using Universal Analysis software V4.5A.

PF-06873600 free base (Form 1) was not hygroscopic; no significant weight gain was observed up to 90% RH and there was no change in solid form after the water sorption study.

TABLE 7
Water Sorption and Desorption Equilibrium Values at 25° C.

Water Content (% w/w)

| Humidity (% RH) | Sorption | Desorption |
|---|---|---|
| 10 | 0.00 | −0.12 |
| 20 | 0.02 | −0.1 |
| 30 | 0.04 | −0.07 |
| 40 | 0.06 | −0.06 |
| 50 | 0.07 | −0.04 |
| 60 | 0.07 | −0.03 |
| 70 | 0.08 | −0.01 |
| 80 | 0.08 | 0.02 |
| 90 | 0.04 | 0.04 |

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention.

1. A crystalline form of 6-(difluoromethyl)-8-[(1R,2R)-2-hydroxy-2-methyl-cyclopentyl]-2-{[1-(methylsulfonyl)piperidin-4-yl]amino}pyrido[2,3-d]pyrimidin-7(8H)-one (PF-06873600) free base, having a powder X-ray diffraction (PXRD) pattern comprising peaks at 2θ values of: 9.6, 18.3 and 22.1 °2θ±0.2 °2θ.

2. The crystalline form of claim 1, having a PXRD pattern further comprising a peak at the 2θ value of: 6.9 °2θ±0.2 °2θ.

3. The crystalline form of claim 1, having a Raman spectrum comprising one or more wavenumber (cm⁻¹) values selected from the group consisting of: 1589, 1626 and 1673 cm⁻¹±2 cm⁻¹.

4. The crystalline form of claim 1, having a ¹³C solid state NMR spectrum comprising one or more resonance (ppm) values selected from the group consisting of: 28.8, 133.2 and 161.4 ppm±0.2 ppm.

5. The crystalline form of claim 1, having a ¹⁹F solid state NMR spectrum comprising a resonance (ppm) value of: −109.6 ppm±0.2 ppm.

6. A crystalline form of PF-06873600 free base, having a ¹⁹F solid state NMR spectrum comprising resonance (ppm) values of: −109.6 and −122.7 ppm±0.2 ppm.

7. The crystalline form of claim 6, having a PXRD pattern comprising one or more peaks at 2θ values selected from the group consisting of: 9.6, 18.3 and 22.1 °2θ±0.2 °2θ.

8. The crystalline form of claim 6, having a Raman spectrum comprising one or more wavenumber (cm⁻¹) values selected from the group consisting of: 1589, 1626 and 1673 cm⁻¹±2 cm⁻¹.

9. The crystalline form of claim 6, having a ¹³C solid state NMR spectrum comprising one or more resonance (ppm) values selected from the group consisting of: 28.8, 133.2 and 161.4 ppm±0.2 ppm.

10. A crystalline form of PF-06873600 free base, having a Raman spectrum comprising wavenumber (cm⁻¹) values of: 1254, 1528, 1589, 1626 and 1673 cm⁻¹±2 cm⁻¹.

11. A crystalline form of PF-06873600 free base, having a ¹³C solid state NMR spectrum comprising resonance (ppm) values of: 28.8, 42.0, 123.0, 133.2 and 161.4 ppm±0.2 ppm.

12. A crystalline form of PF-06873600 free base, having: (a) a powder X-ray diffraction (PXRD) pattern comprising peaks at 2θ values of: 9.6, 18.3 and 22.1 °2θ±0.2 °2θ; (b) a Raman spectrum comprising one or more wavenumber (cm⁻¹) values selected from the group consisting of: 1589, 1626 and 1673 cm⁻¹±2 cm⁻¹; (c) a ¹³C solid state NMR spectrum comprising one or more resonance (ppm) values selected from the group consisting of: 28.8, 133.2 and 161.4 ppm±0.2 ppm; or (d) a ¹⁹F solid state NMR spectrum comprising a resonance (ppm) value of: −109.6 ppm±0.2 ppm; or a combination of two or more of (a), (b), (c) and (d).

13. The crystalline form of claim 1 which is substantially pure and free of alternative forms.

14. The crystalline form of claim 1, wherein the crystalline form is PF-06873600 free base (Form 1).

15. A pharmaceutical composition comprising the crystalline form of PF-06873600 free base according to claim 1, and a pharmaceutically acceptable carrier or excipient.

16. A method of treating abnormal cell growth in a mammal comprising administering to the mammal a therapeutically effective amount of the crystalline form of PF-06873600 free base according to claim 1; wherein the abnormal cell growth is cancer.

17. (canceled)

18. (canceled)

19. (canceled)

20. (canceled)

21. (canceled)

22. The crystalline form of claim 12, having: a PXRD pattern further comprising a peak at 2θ value of 6.9 °2θ±0.2 °2θ; a Raman spectrum further comprising 1528 and 1254 cm⁻¹±2 cm⁻¹; or a ¹⁹F solid state NMR spectrum further comprising −122.7 ppm±0.2 ppm.

23. The crystalline form of claim 12 which is substantially pure and free of alternative forms.

24. The crystalline form of claim 12, wherein the crystalline form is PF-06873600 free base (Form 1).

25. A pharmaceutical composition comprising the crystalline form of PF-06873600 free base according to claim 12, and a pharmaceutically acceptable carrier or excipient.

26. A method of treating abnormal cell growth in a mammal comprising administering to the mammal a therapeutically effective amount of the crystalline form of PF-06873600 free base according to claim 12; wherein the abnormal cell growth is cancer.

27. The method of claim 16, wherein the cancer is mediated by CDK2, CDK4 and/or CDK6.
